(19) United States
(12) Patent Application Publication
(10) Pub. No.: US 2002/0150948 A1
(43) Pub. Date: Oct. 17, 2002

(54) Antibody PTI-HS7 for treatment of alzheimer's disease and other amyloidoses and parkinson's disease

(76) Inventors: Gerardo Castillo, Seattle, WA (US); Paula Y. Choi, Bothell, WA (US); Joel A. Cummings, Seattle, WA (US); Alan D. Snow, Lynnwood, WA (US)

Correspondence Address: Patrick M. Dwyer, Proteo Tech, Inc., 1818 Westlake Avenue N, Suite 114, Seattle, WA, 98109, US

(21) Appl. No.: 10/053,474

(22) Filed: Nov. 2, 2001

Related U.S. Application Data
(60) Provisional application No. 60/245,951, filed on Nov. 3, 2000.

(51) Int. Cl. G01N 33/53

(52) U.S. Cl. 435/7.1

(57) ABSTRACT

A method for diagnosing an amyloid disease, or a susceptibility to an amyloid disease, in a patient, where the disease is related to the levels of a PTI-HS7 antigen in a sample from the patient. The method includes testing with a PTI-HS7 antibody for elevated levels of PTI-HS7 antigen in the patient, whereby any elevated levels of PTI-HS7 antigen are indicative of the presence, susceptibility to, or progression of, the amyloid disease in the patient.

This application claims priority to U.S. provisional patent application No. 60/245951 filed Nov. 11, 2000.

### TECHNICAL FIELD

The invention relates to the identification and use of a specific heparan sulfate GAG moiety antibody known as PTI-HS7 for the diagnosis and therapeutic intervention of Alzheimer's disease, prion diseases, and other amyloid disorders and Parkinson's disease.

### BACKGROUND OF THE INVENTION

During the last few years there has been a heightened interest in the potential role of proteoglycans (PG) and their glycosaminoglycan constituents in the pathogenesis of amyloidosis in general, and in Alzheimer's disease in particular. This is due to a number of important observations made by different laboratories, which have become more aware of the possible significance of these macromolecules in amyloid pathogenesis. Although a number of different studies have cited the existence of different classes of proteoglycans in amyloid deposits, it is becoming clear that HSPGs may be the most important class of PG implicated in Alzheimer's disease and other amyloid disorders.

This is due to the fact that cumulative studies have demonstrated that regardless of the amyloid protein present, the extent of amyloid deposition and the organ involved, HSPGs are a common constituent of all amyloid diseases. In Alzheimer's disease, HSPGs have also been the only class of PG to be immunolocalized to all three characteristic lesions of Alzheimer's disease, including amyloid plaques, neurofibrillary tangles and cerebrovascular amyloid deposits. HSPGs have been shown to play a number of important roles in the pathogenesis of Alzheimer's disease and other amyloidoses, including influencing the processing of amyloid precursor proteins. HSPGs bind to amyloid proteins, enhance amyloid protein aggregation, accelerate amyloid fibril formation, maintain amyloid fibril stability, and protect amyloid from protease degradation. This latter event leads to the hindrance of amyloid clearance.

In the last few years, a number of different HSPGs have now been identified in the characteristic lesions of Alzheimer's disease. These include perlecan, agrin, glypican, and syndecans 1, 2 and 3. It is likely that a number of new HSPGs will be discovered in brain and in the characteristic lesions of Alzheimer's disease in the future However, studies suggest that regardless of the HSPG core protein present, it is the HS GAG chains, probably from different HSPGs, that are likely involved in the pathogenesis of AD and other amyloidoses. This is indicated by a number of different studies which implicate that the GAG chain, and not the core protein, is primarily responsible for binding to the amyloid protein and enhancing amyloid fibril formation.

Problems with developing tools such as antibodies to study the role of specific HS GAG moieties in different diseases stems from the fact that HS, and GAGs in general, are virtually nonimmunogenic, and consequently only a few antibodies have been described. What is needed is an antibody that reliably binds HS and or HS GAGs, and a methodology for using such an antibody in diagnostic and therapeutic applications.

### DISCLOSURE OF THE INVENTION

We have note however the development of a technology which is described in J. Biol. Chem. 27312960-12966, 1998, the text of which is hereby incorporated by this reference as if fully set forth here, that we have novelly adopted to circumvent this problem. The technology described in the cited reference is used for to generate antibodies which recognize specific HS GAG moieties.

Briefly, a single-pot human semisynthetic phage library containing 50 different heavy chain genes each with synthetic random complementary-determining region 3 segments, which are 4-12 amino acid residues in length was used. The library contains >10⁸ clones. Following growth of the library and isolation of phages, heparan sulfate binders were selected by 4 rounds of library panning using HS from bovine kidney. The phage clones expressing the HS binding antibodies were then screened for unique antibody inserts by PCR, fingerprinting and sequencing. 19 clones expressing anti-HS antibodies and containing full-length DNA inserts were isolated. The specificity of the different anti-HS antibodies were evaluated by ELISAs and immunofluorescent studies.

We have tested a number of these anti-HS antibodies using Alzheimer's disease brain tissue as a target. One of the most immunoreactive of the antibodies, in all amyloid diseases tested, is an antibody derived from the clone known in the library as HS4C3, which we have adopted and herein refer to as PTI-HS7. Testing demonstrated that the PTI-HS7 antibody shows no reactivity with chondroitin-4-sulfate, chondroitin-6-sulfate, dermatan sulfate, keratan sulfate, dextran sulfate, hyaluronic acid, DNA, bovine serum albumin or polystyrene.

Remarkably, it does demonstrate major reactivity with heparin, and the reactivity is abolished when HS or heparin has all of its sulfate removed, or either its N or O-sulfate removed. In addition, treatment of HS with heparinase I, II, or III or with nitrous acid destroys reactivity. These initial studies suggest that sulfate, and particularly O- and N-sulfate in HS/heparin GAGs are important for PTI-HS7 antibody recognition. It does appear therefore that we have discovered an antibody that reliably binds HS and or HS GAGs, and a methodology for using such an antibody in diagnostic and therapeutic applications against the amyloid disease, and against Parkinson's disease and other α-synuclein diseases, is disclosed herein.

The methods we employed included use of the PTI-HS7 antibody for immunostaining of a number of different cases including Alzheimer's disease, prion diseases (including GSS and C.D.), and systemic/organ amyloidosis (including AA, AL and beta₂-microglobulin amyloids). Amyloid deposits were verified by Congo red staining as viewed under polarized light or by immunostaining with specific amyloid protein antibodies such as 6E10 for Alzheimer's disease Aβ. The specificity of immunostaining with PTI-HS7 was demonstrated by abolishment of PTI-HS7 immunostaining in the presence of excess heparin.

As a result of our studies, we believe that PTI-HS7 is a specific HS antibody that recognize a heparin-like moiety consisting of 6-O-sulfated glucosamine sulfate and 2-O-sulfated iduronic acid. We used PTI-HS7 to detect the presence in Alzheimer's disease, prion diseases and other systemic/organ amyloid diseases of this moiety.

Anti-PTI-HS7 recognized a specific HS moiety that is present in all three characteristic lesions of Alzheimer's disease, including amyloid plaques (both diffuse and core plaques), neurofibrillary tangles and cerebrovascular amyloid deposits. The PTI-HS7 epitope was also found to be a common constituent of other amyloids including prion diseases (GSS, C.D.), and systemic/organ amyloidoses (AA, AL and beta₂-microglobulin deposits). The presence of a common HS GAG moiety in different amyloid diseases regardless of the amyloid protein present, extent of amyloid accumulation, or the organ involved suggests that specific HS GAG moieties are involved in the pathogenesis of these diseases.

This surprising discovery has implications for the use of the PTI-HS7 antibody as a diagnostic tool for the identification of lesions in Alzheimer's disease and other amyloidoses, as well as implications for the use of the PTI-HS7 antibody as a therapeutic agent for the prevention of amyloid formation, deposition, accumulation and persistence in Alzheimer's disease, prion diseases, systemic/organ amyloid diseases, and diseases involving the formation of tangles.

We have thus determined that a specific heparan sulfate GAG moiety is implicated in Alzheimer's Disease, prion diseases and other amyloid disorders, by using the phage display generated antibody PTI-HS7. We have also determined that the same heparan sulfate GAG moiety is present in different amyloid deposits regardless of the nature of the amyloid protein present, establishing that a specific HS GAG moiety is a common constituent of all amyloids.

Antibody PTI-HS7 was also found to immunostain basement membranes of the glomerulus and pertibular capillaries in kidney which was precluded by incubation of the antibody with HS or heparin. A strong immunoreaction was observed with HS from bovine kidney, but not HS from bovine aorta. Since kidney HS is high in O-sulfation, and aorta HS is lower in O-sulfation, these studies suggest that O-sulfation is preferred for epitope recognition over N-sulfate. Based on all of these observations, it is concluded that the PTI-HS7 antibody most likely recognizes a heparin-like sequence consisting of disaccharides formed by 6-O-sulfated glucosamine sulfate and 2-O-sulfate iduronic acid.

Antibody PTI-HS7 is believed to recognize a heparin-like glycosaminoglycan (GAG) moiety containing both O- and N-sulfate, was found to immunolocalize to all 3 characteristic lesions of Alzheimer's disease (amyloid plaques, neurofibrillary tangles and cerebrovascular amyloid deposits) and to the amyloid deposits of prion diseases (including Creutzfeldt-Jakob disease and Gerstmann-Straussler syndrome), and systemic/organ amyloidosis (including AA amyloid, AL amyloid and beta₂microglobulin amyloidosis). This antibody may therefore be used to detect and identify amyloid deposits and tangle inclusions in a variety of amyloid diseases and has use as a diagnostic marker for amyloid deposits. In addition, this antibody may be used for therapeutic intervention to prevent the accumulation of heparan sulfate GAGs and associated amyloid deposits. Heparan sulfate GAGs have been previously demonstrated to play an important role in amyloid fibril formation, deposition and persistence in all amyloid diseases, and in disorders demonstrating neurofibrillary tangle accumulation.

Methods are disclosed herein for the treatment and diagnosis of Alzheimer's disease and other amyloid disorders. Methods are disclosed for treating amyloid disorders, comprising administering to a subject or patient a therapeutically effective dose of anti-PTI-HS7 antibody.

The PTI-HS7 antibody of the present invention may be prepared by known immunological methods or by biotechnological methods known to those skilled in the art. Assays useful for the screening and identification of amyloid diseases in patients using the anti-PTI-HS7 antibody are also disclosed. In addition, methods are disclosed for the labeling of anti-PTI-HS7 antibody derived from the invention for diagnosis of Alzheimer's and other amyloidoses.

It is an object of the present invention is to provide a method for treating Alzheimer's disease and other amyloid disorders involving the formation and persistence of amyloid proteins.

The invention also discloses methods to utilize the PTI-HS7 antibody as diagnostic or imaging agents for amyloidoses.

The invention also discloses methods to utilize the PTI-HS7 antibody for the treatment of Alzheimer's disease and other amyloid disorders.

A primary object of the present invention is to establish new therapeutic methods for Alzheimer's disease and other Aβ amyloid diseases. These Aβ diseases include, but are not limited to, the amyloid associated with Alzheimer's disease and Down's syndrome, and various forms of cerebral amyloidosis, known to those knowledgeable in the art.

A primary object of the present invention is to use PTI-HS7 antibody as potent inhibitors of amyloid formation, deposition, accumulation and/or persistence in Alzheimer's disease and other amyloidoses.

Yet another aspect of the present invention is to make use of PTI-HS7 antibody, as potential therapeutics to inhibit the deposition, formation and accumulation of fibrillar amyloid in Alzheimer's disease and other amyloidosis disorders, and to enhance the clearance and/or removal of pre-formed amyloid deposits.

Yet another object of the present invention is to use the PTI-HS7 antibody as described herein as a specific indicator for the presence and extent of heparan sulfate (HS) and its associated amyloid accumulation and breakdown in brain by monitoring biological fluids including, but not limited to, cerebrospinal fluid, blood, serum, urine, saliva, sputum and stool.

Yet another object of the present invention is to make use of the PTI-HS7 antibody as described herein, as potential blocking therapeutics for the interaction of heparan sulfate and amyloid proteins in a number of biological processes and diseases (such as in Alzheimer's disease, Down's syndrome and other amyloid diseases).

Yet another object of the present invention is to provide compositions and methods involving administering to a subject a therapeutic dose of a PTI-HS7 antibody, which inhibit amyloid deposition. Accordingly, the compositions and methods of the invention are useful for inhibiting amyloidosis in disorders in which amyloid deposition occurs. The PTI-HS7 antibody of the invention can be used therapeutically to treat amyloidosis or can be used prophylactically in a subject susceptible to amyloidosis. The methods of the invention are based, at least in part, in directly inhibiting heparan sulfate-amyloid protein interactions which will cause the breakdown of amyloid deposits and retard the growth/accumulation of new amyloid deposits.

Yet another object of the present invention is to provide pharmaceutical compositions for treating amyloidoses. The pharmaceutical compositions include a therapeutic compound of the invention in an amount effective to inhibit amyloid deposition in a pharmaceutically acceptable vehicle.

Another aspect of the invention is to provide new and novel monoclonal PTI-HS7 antibodies which can be utilized in a number of in vitro assays to specifically detect HS-binding amyloid proteins or protein fragments in human tissues and/or biological fluids. Monoclonal PTI-HS7 antibodies can be utilized to detect and quantify amyloid disease in human tissues and/or biological fluids. Monoclonal antibodies may be prepared as described herein or by standard techniques known to those skilled in the art.

Another object of the present invention is to use the PTI-HS7 antibody referred to above, for the detection and specific localization of heparan sulfate important in the amyloid diseases in human tissues, cells, and/or cell culture using standard immunohistochemical techniques.

Yet another aspect of the present invention is to use antibodies recognizing heparan sulfate or portions thereof, for in vivo labeling; for example, with a radionucleotide, for radioimaging to be utilized for in vivo diagnosis, and/or for in vitro diagnosis.

Yet another object of the present invention is to use the PTI-HS7 antibody of the present invention in each of the various therapeutic and diagnostic applications described above.

Another object of the invention is to provide monoclonal PTI-HS7 antibodies that can be utilized in a number of in vitro assays to specifically detect amyloid protein-associated heparan sulfate in human tissues and/or biological fluids. Monoclonal antibodies may be prepared by standard techniques by one skilled in the art.

Yet another object of the present invention is to use PTI-HS7 antibodies as described herein as a specific indicator for the presence and extent of heparan sulfate and associated amyloid formation, deposition, accumulation and/or persistence by monitoring biological fluids including, but not limited to, cerebrospinal fluid, blood, serum, urine, saliva, sputum, and stool.

Preferred pharmaceutical agents for treating amyloidosis in a patient have a therapeutically effective amount of PTI-HS7 antibodies, and having an amyloid inhibitory activity or efficacy greater than 30%, as compared to duly established controls, such as patients who do not received the preferred pharmaceutical agent.

An important Aβ amyloidosis to which the disclosed therapeutics are addressed is Alzheimer's disease. A preferred therapeutically effect amount of disclosed PTI-HS7 antibody is a dosage in the range of from about 10 μg of protein or glycosaminoglycan (by weight) to about 50 mg/kg body weight/per day, and more preferably in the range of from about 100 μg to about 10 mg/kg body weight per day.

The pharmaceutical agent may advantageously be administered in a parenterally injectable or infusible form or orally.

A method is also disclosed to diagnose a disease or susceptibility to amyloidosis related to the level of PTI-HS7 antigen, as detected by anti-PTI-HS7 antibodies. First the levels of PTI-HS7 antigen in a sample are determined, whereby the levels are indicative of the presence of heparan sulfate associated amyloid deposits, susceptibility to amyloidosis, or progression of amyloidosis.

The sample assayed may be a biological fluid, and the biological fluid may be serum derived from humans.

In a particular embodiment there is a method of using a specific heparan sulfate glycosaminoglycan antibody identified herein as PTI-HS7 to diagnose the presence of amyloid deposits, or Parkinson's disease, in a human patient.

In addition there is a method for diagnosing an amyloid disease, or a susceptibility to an amyloid disease, in a patient, where the disease is related to the levels of a PTI-HS7 antigen. The method includes testing with a PTI-HS7 antibody for elevated levels of PTI-HS7 antigen in the patient, preferably in a biological fluid of the patient, whereby any elevated levels of PTI-HS7 antigen are indicative of the presence, susceptibility to, or progression of, the amyloid disease in the patient. The biological fluid may be any or all of blood, plasma, serum, cerebrospinal fluid, sputum, saliva, urine and stool. Alternatively, tissue section of the patient may be tested by standard immunohistochemistry.

The particular amyloid disease tested for will typically have an associated amyloid and include such amyloid disease as Alzheimer's disease, Down's syndrome, hereditary cerebral hemorrhage with amyloidosis of the Dutch type, the amyloid disease associated with type II diabetes, the amyloid disease associated with chronic inflammation, various forms of malignancy and Familial Mediterranean Fever, the amyloid disease associated with multiple myeloma and other B-cell dyscrasias, the amyloid disease associated with the prion diseases including Creutzfeldt-Jakob disease, Gerstmann-Straussler syndrome, kuru and animal scrapie, the amyloid disease associated with long-term hemodialysis and carpal tunnel syndrome, the amyloid disease associated with endocrine tumors such as medullary carcinoma of the thyroid, and the alpha-synuclein associated diseases including Parkinson's disease and Lewy body disease.

Associated amyloids include beta-amyloid protein or Aβ, AA amyloid or inflammation-associated amyloid, AL amyloid, amylin or islet amyloid polypeptide, PrP amyloid, beta₂-microglobulin amyloid, transthyretin or prealbumin, or variants of procalcitonin.

Another method presented is for the treatment of a patient having an identified clinical need to interfere with the pathological effects of amyloid deposits, and includes administering to the patient a therapeutically effective amount of PTI-HS7 antibody.

In a method for detection of amyloid disease in a human patient, a radiolabeled PTI-HS7 antibody is used for radioimaging or in vivo diagnosis.

There is also a method for detection and quantization of amyloid associated HS7 antigen in biological fluids. The preferred steps include

a) binding an anti-PTI-HS7 monoclonal antibody to a microtiter well;

b) blocking the microtiter well with Tris-buffered saline or the like containing detergent plus bovine serum albumin;

c) adding to the well a quantity of biological fluid selected from the group of fluids consisting of cerebrospinal fluid, blood, plasma, serum, urine, sputum, saliva, urine and stool;

d) incubating the biological fluid in the microtiter well to bind the antibody;

e) adding to the microtiter well a second labeled monoclonal antibody against the same HS7 antigen but which is against a different epitope to bind to any HS7 protein captured by the first antibody;

f) incubating with a substrate until a significant color reaction develops to detect antibody bound materials.

There is also a method of treatment, prevention or management of an amyloidosis, or a disease related to alpha-synuclein, in a mammalian subject susceptible to, or afflicted by, the amyloidosis or alpha-synuclein disease. The method includes the step of administering to the subject a therapeutic amount of PTI-HS7 antibody.

In a method for diagnosing an amyloid disease in a subject, the method includes determining a level of PTI-HS7 antigen in a sample from the subject and comparing the level of PTI-HS7 antigen in the sample to normal levels, wherein an increase in the level of PTI-HS7 antigen in the sample relative to normal indicates the amyloid disease.

A new method is disclosed for determining whether a subject is at increased risk of developing an amyloid disease, or diagnosing or prognosing the amyloid disease in the subject, or monitoring a progression of the amyloid disease in the subject. The important steps are: a) determining a level of PTI-HS7 antigen in a sample from the subject; and b) comparing the level of PTI-HS7 antigen in the sample to a reference value representing a known disease or health status; thereby determining whether the subject is at increased risk of developing the amyloid disease, or diagnosing or prognosing the amyloid disease in the subject, or monitoring the progression of the amyloid disease in the subject.

There is also a method of evaluating a treatment for an amyloid disease that includes determining a level of PTI-HS7 antigen in a sample from a subject under the treatment; and comparing the level of PTI-HS7 antigen in the sample to a reference value representing a known disease or health status; thereby evaluating the treatment for the amyloid disease. A biological fluid is preferably tested, preferably cerebrospinal fluid.

In the diagnostic methods above, an increase of level of PTI-HS7 antigen in the sample from the subject relative to a reference value representing a known amyloid disease or health status indicates the diagnosis or prognosis, or progression, or increased risk of the amyloid disease. The level of PTI-HS7 antigen in a sample is advantageously detected and quantified using an immunoassay and/or a binding assay.

The reference value referred to is preferably that of a level of PTI-HS7 antigen in a sample from a subject not afflicted with the amyloid disease, or some other published reference value related to the absence of the pathology tested for. Alternatively reference may be had to the patient herself by comparing the level of PTI-HS7 antigen in the sample from the subject with a level of PTI-HS7 antigen in a series of samples from the subject taken over a period of time to establish progress or decline of the patient as to the disease.

A kit is also presented for determining increased risk of developing an amyloid disease, or diagnosing or prognosing the amyloid disease in a subject, or monitoring a progression of the amyloid disease in the subject. The kit includes a reagent for detecting the presence or absence of PTI-HS7 antigen, the reagent comprising PTI-HS7 antibody, and instructions for determining increased risk of developing an amyloid disease, or diagnosing or prognosing the amyloid disease in a subject, or monitoring a progression of the amyloid disease in the subject by detecting and/or quantifying a level of PTI-HS7 antigen in a sample from a subject, and determining whether the subject is at increased risk of developing the amyloid disease, or diagnosing or prognosing the amyloid disease in a subject, or monitoring the progression of the amyloid disease in the subject, wherein a varied level of PTI-HS7 antigen in a sample relative to a reference value representing a known disease or health status is indicates the increased risk of developing an amyloid disease, or the diagnosis or prognosis of the amyloid disease in the subject, or the progression of the amyloid disease in the subject.

### BRIEF DESCRIPTION OF THE DRAWINGS

FIG. 1 shows photomicrographs of PTI-HS7 antibody immunostaining in Alzheimer's disease brain.

FIG. 2 demonstrates the specificity of the PTI-HS7 antibody immunostaining.

FIG. 3 demonstrates that the HS epitope recognized by the PTI-HS7 antibody is also present in cerebrovascular deposits in Alzheimer's disease brain.

FIG. 4 illustrates the immunostaining of prion protein amyloid plaques in cases of Gerstmann-Struasller Syndrome (GSS) and Creutzfeldt-Jakob Disease (C.D.) by the PTI-HS7 antibody.

FIG. 5 demonstrates the presence of PTI-HS7 immunoreactivity localized to amyloid deposits from a case of AA amyloidosis.

FIG. 6 demonstrates the presence of PTI-HS7 immunoreactivity localized to amyloid deposits from a case of AL and beta₂-microglobulin amyloidosis.

### BEST MODE OF CARRYING OUT THE INVENTION

Amyloid and Amyloidosis

Amyloid is a generic term referring to a group of diverse but specific extracellular protein deposits which all have common morphological properties, staining characteristics, and X-ray diffraction spectra. Regardless of the nature of the amyloid protein deposited all amyloids have the following characteristics: 1) showing an amorphous appearance at the light microscopic level, appearing eosinophilic using hematoxylin and eosin stains; 2) staining with Congo red and demonstrating a red/green birefringence as viewed under polarized light (Puchtler et al., J. Histochem. Cytochem. 10:355-364, 1962), 3) containing a predominant beta-pleated sheet secondary structure, and 4) ultrastructurally consisting of non-branching fibrils of indefinite length and with a diameter of 7-10 nm.

Amyloidoses today are classified according to the specific amyloid protein deposited. The amyloids include, but are not limited to, the amyloid associated with Alzheimer's disease, Down's syndrome and hereditary cerebral hemorrhage with amyloidosis of the Dutch type (where the specific amyloid is referred to as betaamyloid protein or Aβ), the amyloid associated with chronic inflammation, various forms of malignancy and familial Mediterranean fever (where the specific amyloid is referred to as AA amyloid or inflammation-associated amyloid), the amyloid associated with multiple myeloma and other B-cell dyscrasias (where the specific amyloid is referred to as AL amyloid), the amyloid associated with type II diabetes (where the specific amyloid is referred to as amylin or islet amyloid), the amyloid associated with the prion diseases including Creutzfeldt-Jakob disease, Gerstmann-Straussler syndrome, kuru, and scrapie (where the specific amyloid is referred to as PrP amyloid), the amyloid associated with long-term hemodialysis and carpal tunnel syndrome (where the specific amyloid is referred to as beta₂-microglobulin amyloid), the amyloid associated with senile cardiac amyloid and familial amyloidotic polyneuropathy (where the specific amyloid is referred to as prealbumin or transthyretin amyloid), and the amyloid associated with endocrine tumors such as medullary carcinoma of the thyroid (where the specific amyloid is referred to as variants of procalcitonin).

Although amyloid deposits in clinical conditions share common physical properties relating to the presence of a beta-pleated sheet conformation, it is now clear that many different chemical types exist and additional ones are likely to be described in the future. It is currently thought that there are several common pathogenetic mechanisms that may be operating in amyloidosis in general. In many cases, a circulating precursor protein may result from overproduction of either intact or aberrant molecules (for example, in plasma cell dyscrasias), reduced degradation or excretion (serum amyloid A in some secondary amyloid syndromes and beta₂microglobulin in long-term hemodialysis), or genetic abnormalities associated with variant proteins (for example, familial amyloidotic polyneuropathy). Proteolysis of a larger protein precursor molecule occurs in many types of amyloidosis, resulting in the production of lower molecular weight fragments that polymerize and assume a beta-pleated sheet conformation as tissue deposits, usually in an extracellular location. The precise mechanisms involved and the aberrant causes leading to changes in proteolytic processing and/or translational modification are not known in most amyloids.

Systemic amyloids which include the amyloid associated with chronic inflammation, various forms of malignancy and familial Mediterranean fever (i.e. AA amyloid or inflammation-associated amyloidosis) (Benson and Cohen, Arth. Rheum. 22:36-42, 1979; Kamei et al, Acta Path. Jpn. 32:123-133, 1982; McAdam et al., Lancet 2:572-573, 1975; Metaxas, Kidney Int. 20:676-685, 1981), and the amyloid associated with multiple myeloma and other B-cell dyscrasias (i.e. AL amyloid) (Harada et al., J. Histochem. Cytochem. 19:1-15, 1971), as examples, are known to involve amyloid deposition in a variety of different organs and tissues generally lying outside the central nervous system. Amyloid deposition in these diseases may occur, for example, in liver, heart, spleen, gastrointestinal tract, kidney, skin, and/or lungs (Johnson et al, N. Engl. J. Med. 321:513-518, 1989). For most of these amyloidoses, there is no apparent cure or effective treatment and the consequences of amyloid deposition can be detrimental to the patient. For example, amyloid deposition in the kidney may lead to renal failure, whereas amyloid deposition in the heart may lead to heart failure. For these patients, amyloid accumulation in systemic organs leads to eventual death generally within 3-5 years. Other amyloidoses may affect a single organ or tissue such as observed with the AB amyloid deposits found in the brains of patients with Alzheimer's disease and Down's syndrome: the PrP amyloid deposits found in the brains of patients with Creutzfeldt-Jakob disease, Gerstmann-Straussler syndrome, and kuru; the islet amyloid (amylin) deposits found in the islets of Langerhans in the pancreas of 90% of patients with type II diabetes (Johnson et al, N. Engl. J. Med. 321:513-518, 1989; Lab. Invest. 66:522 535, 1992); the beta₂-microglobulin amyloid deposits in the medial nerve leading to carpal tunnel syndrome as observed in patients undergoing long-term hemodialysis (Geyjo et al, Biochem. Biophys. Res. Comm. 129:701-706, 1985; Kidney Int. 30:385-390, 1986); the prealbumin/transthyretin amyloid observed in the hearts of patients with senile cardiac amyloid; and the prealbumin/transthyretin amyloid observed in peripheral nerves of patients who have familial amyloidotic polyneuropathy (Skinner and Cohen, Biochem. Biophys. Res. Comm. 99:1326-1332, 1981; Saraiva et al, J. Lab. Clin. Med. 102:590-603, 1983; J. Clin. Invest. 74:104-119, 1984; Tawara et al, J. Lab. Clin. Med. 98:811-822, 1989).

Alzheimer's Disease and the Aging Population

Alzheimer's disease is a leading cause of dementia in the elderly, affecting 5-10% of the population over the age of 65 years (A Guide to Understanding Alzheimer's Disease and Related Disorders, Jorm, ed., New York University Press, New York, 1987). In Alzheimer's disease, the parts of the brain essential for cognitive processes such as memory, attention, language, and reasoning degenerate, robbing victims of much that makes us human, including independence. In some inherited forms of Alzheimer's disease, onset is in middle age, but more commonly, symptoms appear from the mid-60's onward. Alzheimer's disease today affects 4-5 million Americans, with slightly more than half of these people receiving care at home, while the others are in many different health care institutions. The prevalence of Alzheimer's disease and other dementias doubles every 5 years beyond the age of 65, and recent studies indicate that nearly 50% of all people age 85 and older have symptoms of Alzheimer's disease (1999 Progress Report on Alzheimer's Disease, National Institute on Aging/National Institute of Health). 13% (33 million people) of the total population of the United States are age 65 and older, and this percentage will climb to 20% by the year 2025 (1999 Progress Report on Alzheimer's Disease).

Alzheimer's disease also puts a heavy economic burden on society. A recent study estimated that the cost of caring for one Alzheimer's disease patient with severe cognitive impairments at home or in a nursing home, is more than $47,000 per year (A Guide to Understanding Alzheimer's Disease and Related Disorders). For a disease that can span from 2 to 20 years, the overall cost of Alzheimer's disease to families and to society is staggering. The annual economic toll of Alzheimer's disease in the United States in terms of health care expenses and lost wages of both patients and their caregivers is estimated at $80 to $100 billion (1999 Progress Report on Alzheimer's Disease).

Tacrine hydrochloride (“Cognex”), the first FDA approved drug for Alzheimer's disease, is a acetylcholinesterase inhibitor (Cutler and Sramek, N. Engl. J. Med. 328:808 810, 1993). However, this drug has showed limited success in producing cognitive improvement in Alzheimer's disease patients and initially had major side effects such as liver toxicity. The second more recently FDA approved drug, donepezil (“Aricept”), which is also an acetylcholinesterase inhibitor, is more effective than tacrine, by demonstrating slight cognitive improvement in Alzheimer's disease patients (Barner and Gray, Ann. Pharmacotherapy 32:70-77, 1998; Rogers and Friedhoff, Eur. Neuropsych. 8:67-75, 1998), but is not believed to be a cure. Therefore, it is clear that there is a need for more effective treatments for Alzheimer's disease patients.

Amyloid as a Therapeutic Target for Alzheimer's Disease

Alzheimer's disease is characterized by the deposition and accumulation of a 39-43 amino acid peptide termed the beta-amyloid protein, Aβ or β/A4 (Glenner and Wong, Biochem. Biophys. Res. Comm. 120:885-890, 1984; Masters et al., Proc. Natl. Acad. Sci. USA 82:4245-4249, 1985; Husby et al., Bull. WHO 71:105-108, 1993). Aβ is derived by protease cleavage from larger precursor proteins termed beta-amyloid precursor proteins (or βPPs) of which there are several alternatively spliced variants. The most abundant forms of the BPPs include proteins consisting of 695, 751 and 770 amino acids (Tanzi et al., Nature 331:528-530, 1988; Kitaguchi et al., Nature 331:530532, 1988; Ponte et al., Nature 331:525-527, 1988).

The small Aβ peptide is a major component which makes up the amyloid deposits of “plaques” in the brains of patients with Alzheimer's disease. In addition, Alzheimer's disease is characterized by the presence of numerous neurofibrillary “tangles”, consisting of paired helical filaments which abnormally accumulate in the neuronal cytoplasm (Grundke-Iqbal et al., Proc. Natl. Acad. Sci. USA 83:4913-4917, 1986; Kosik et al., Proc. Natl. Acad. Sci. USA 83:4044-4048, 1986; Lee et al., Science 251:675-678, 1991). The pathological hallmark of Alzheimer's disease is therefore the presence of “plaques” and “tangles”, with amyloid being deposited in the central core of the plaques. The other major type of lesion found in the Alzheimer's disease brain is the accumulation of amyloid in the walls of blood vessels, both within the brain parenchyma and in the walls of meningeal vessels which lie outside the brain. The amyloid deposits localized to the walls of blood vessels are referred to as cerebrovascular amyloid or congophilic angiopathy (Mandybur, J. Neuropath. Exp. Neurol. 45:79-90, 1986; Pardridge et al., J. Neurochem. 49:1394-1401, 1987).

For many years there has been an ongoing scientific debate as to the importance of “amyloid” in Alzheimer's disease, and whether the “plaques” and “tangles” characteristic of this disease were a cause or merely a consequence of the disease. Within the last few years, studies now indicate that amyloid is indeed a causative factor for Alzheimer's disease and should not be regarded as merely an innocent bystander. The Alzheimer's Aβ protein in cell culture has been shown to cause degeneration of nerve cells within short periods of time (Pike et al., Br. Res. 563:311-314, 1991; J. Neurochem. 64:253-265, 1995). Studies suggest that it is the fibrillar structure (consisting of a predominant beta-pleated sheet secondary structure), characteristic of all amyloids, that is responsible for the neurotoxic effects. Aβ has also been found to be neurotoxic in slice cultures of hippocampus (Harrigan et al., Neurobiol. Aging 16:779-789, 1995) and induces nerve cell death in transgenic mice (Games et al., Nature 373:523-527, 1995; Hsiao et al., Science 274:99-102, 1996). Injection of the Alzheimer's Aβ into rat brain also causes memory impairment and neuronal dysfunction (Flood et al., Proc. Natl. Acad. Sci. USA 88:3363-3366, 1991; Br. Res. 663:271-276, 1994).

Probably, the most convincing evidence that Aβ amyloid is directly involved in the pathogenesis of Alzheimer's disease comes from genetic studies. It has been discovered that the production of Aβ can result from mutations in the gene encoding, its precursor, beta amyloid precursor protein (Van Broeckhoven et al., Science 248:1120-1122, 1990; Murrell et al., Science 254:97-99, 1991; Haass et al., Nature Med. 1:1291-1296, 1995). The identification of mutations in the beta-amyloid precursor protein gene which causes early onset familial Alzheimer's disease is the strongest argument that amyloid is central to the pathogenetic process underlying this disease. Four reported disease-causing mutations have now been discovered which demonstrate the importance of Aβ in causing familial Alzheimer's disease (reviewed in Hardy, Nature Genet. 1:233-234, 1992). All of these studies suggest that providing a drug to reduce, eliminate or prevent fibrillar Aβ formation, deposition, accumulation and/or persistence in the brains of human patients will serve as an effective therapeutic.

Discovery and identification of new compounds or agents as potential therapeutic agents to arrest amyloid deposition, accumulation and/or persistence that occurs in Alzheimer's disease and other amyloidoses are desperately sought.

Parkinson's Disease and α-Synuclein Fibril Formation

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of (α-Synuclein (Spillantini et al., Proc. Natl. Acad. Sci. USA 95:6469-6473, 1998; Arai et al., Neurosc. Lett. 259:83-86, 1999), an 140-amino acid protein (Ueda et al., Proc. Natl. Acad. Sci. USA 90:11282-11286, 1993). Two dominant mutations in (α-Synuclein causing familial early onset Parkinson's disease have been described suggesting that Lewy bodies contribute mechanistically to the degeneration of neurons in Parkinson's disease (Polymeropoulos et al., Science 276:2045-2047, 1997; Kruger et al., Nature Genet. 18:106-108, 1998). Recently, in vitro studies have demonstrated that recombinant (α-Synuclein can indeed form Lewy body-like fibrils (Conway et al., Nature Med. 4:1318-1320, 1998; Hashimoto et al., Brain Res. 799:301306, 1998; Nahri et al., J. Biol. Chem. 274:9843-9846, 1999). Most importantly. both Parkinson's disease-linked α-Synuclein mutations accelerate this aggregation process which suggests that such in vitro studies may have relevance for Parkinson's disease pathogenesis. (α-Synuclein aggregation and fibril formation fulfills of the criteria of a nucleation-dependent polymerization process (Wood et al., J. Biol. Chem. 274:19509-19512, 1999). In this regard (α-Synuclein fibril formation resembles that of Alzheimer's beta-amyloid protein (Aβ) fibrils. (α-Synuclein recombinant protein, and non-amyloid component (known as NAC-P), which is a 35-amino acid peptide fragment of (α-Synuclein, both have the ability to form fibrils when incubated at 37° C., and are positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. USA 90:11282-11286, 1993).

Parkinson's disease (α-Synuclein fibrils, like the AB fibrils of Alzheimer's disease, also consist of a predominant beta-pleated sheet structure. We believe, therefore, that compounds found to inhibit Alzheimer's disease Aα amyloid fibril formation are also effective in the inhibition of (α-Synuclein fibril formation. These compounds also serve as therapeutics for Parkinson's disease, in addition to having efficacy as a therapeutic for Alzheimer's disease and other amyloid disorders.

The disclosures of these and other documents referred to throughout this application are incorporated herein by reference.

The drawings discussed below are illustrative of embodiments of the invention and are not meant to limit the scope of the invention.

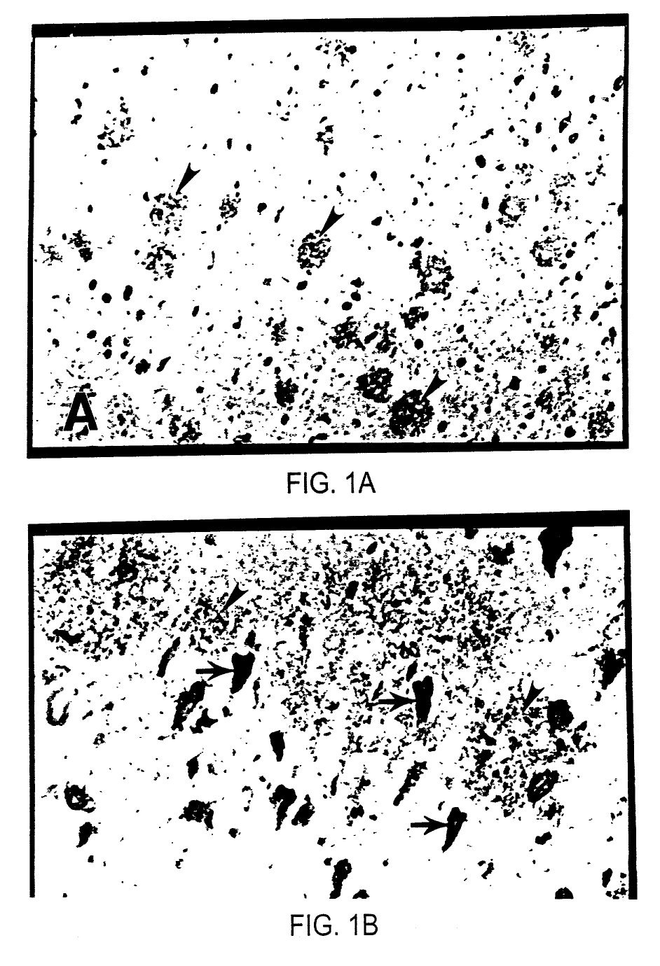

FIG. 1 shows photomicrographs of PTI-HS7 antibody immunostaining in Alzheimer's disease brain. As shown in Figure A, strong immunostaining of amyloid plaques (arrowheads) is observed. In Figure B, a similar strong immunoreactivity for both diffuse plaques (arrowheads) and neurofibrillary tangles (arrows) is observed

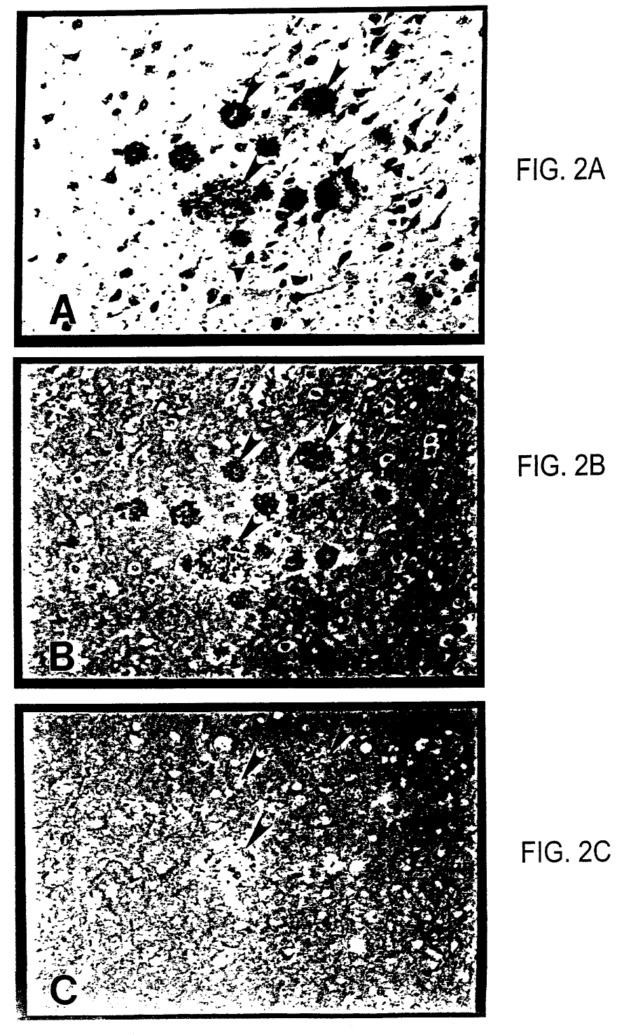

FIG. 2 demonstrates the specificity of the PTI-HS7 antibody immunostaining. Serial sections immunostained with 6E10 antibody, as shown in Figure A, and the PTI-HS7 antibody as shown in Figure B, demonstrate the co-localization of PTI-HS7 epitopes to Alzheimer's AB deposits (arrowheads). In addition, Figure C demonstrates the specificity of the HS epitope recognized whereby the adjacent serial section demonstrates virtual abolishment of PTI-HS7 immunostaining (arrowheads) when the same antibody is used in the presence of excess heparin.

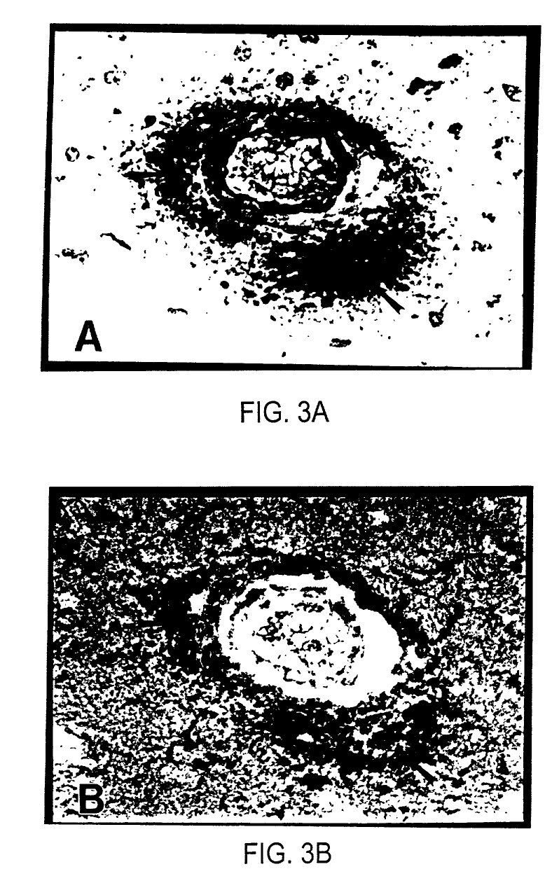

FIG. 3 demonstrates that the HS epitope recognized by the PTI-HS7 antibody is also present in cerebrovascular deposits in Alzheimer's disease brain. Figure A demonstrates the presence of Aβ (arrows) as shown by positive 6E10 antibody immunostaining. Figure B is a serial section immunostained with PTI-HS7 and demonstrates co-localization of HS GAGs to Aβ deposits (arrows).

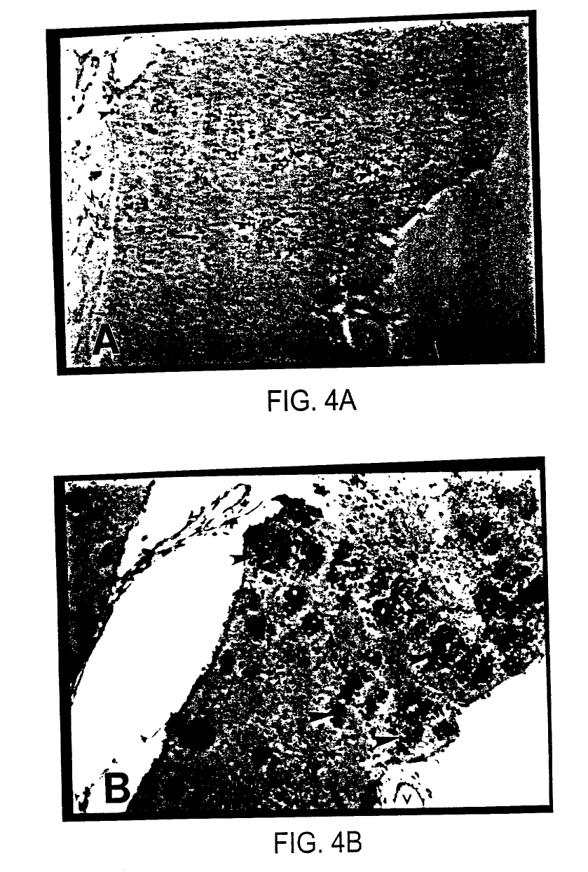

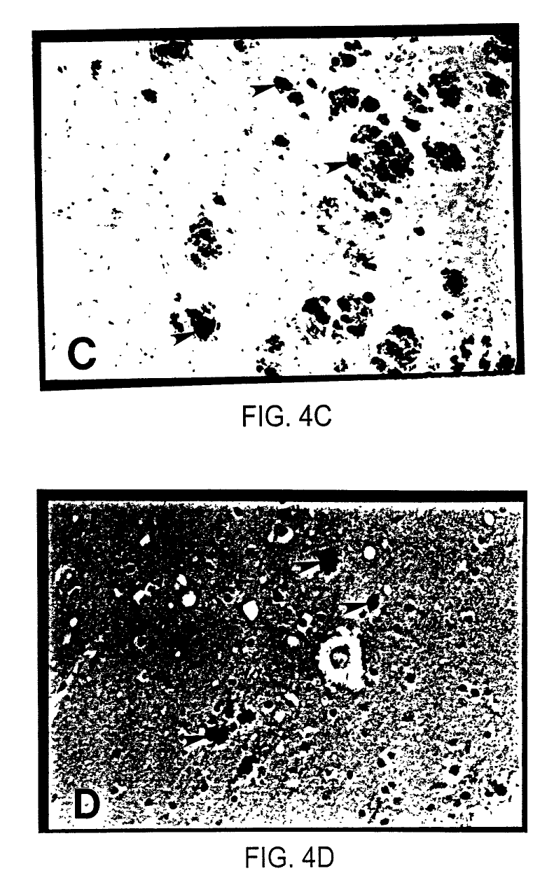

FIG. 4 illustrates the immunostaining of prion protein amyloid plaques in cases of Gerstmann-Struasller Syndrome (GSS) and Creutzfeldt-Jakob Disease (C.D.) by the PTI-HS7 antibody. Figure A demonstrates GSS amyloid plaques in the cerebellum stained with Congo red and viewed under polarized light (arrowheads). Figures B and C demonstrate strong immunoreactivity with the PTI-HS7 antibody in GSS amyloid plaques (arrowheads). As shown in Figure D, positive PTI-HS7 immunoreactivity is localized to amyloid plaques (arrowheads) in the cerebellum of a patient who died with confirmed C.D.

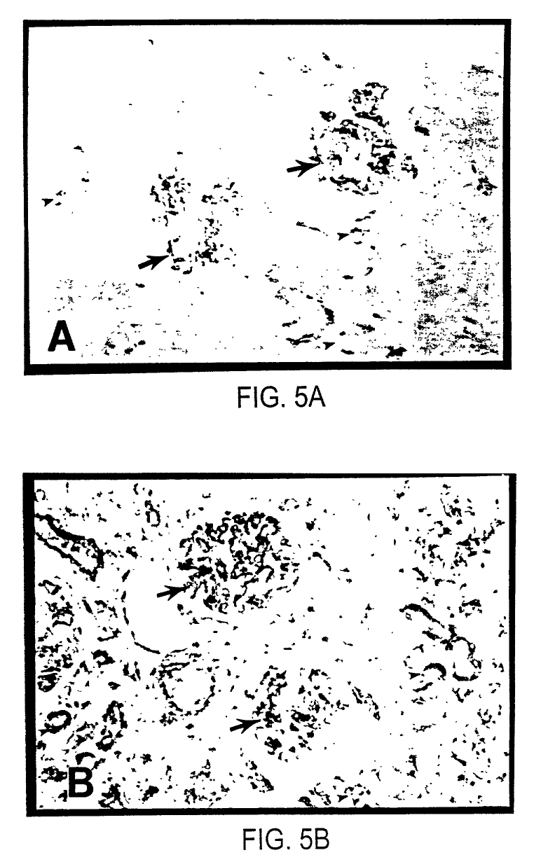

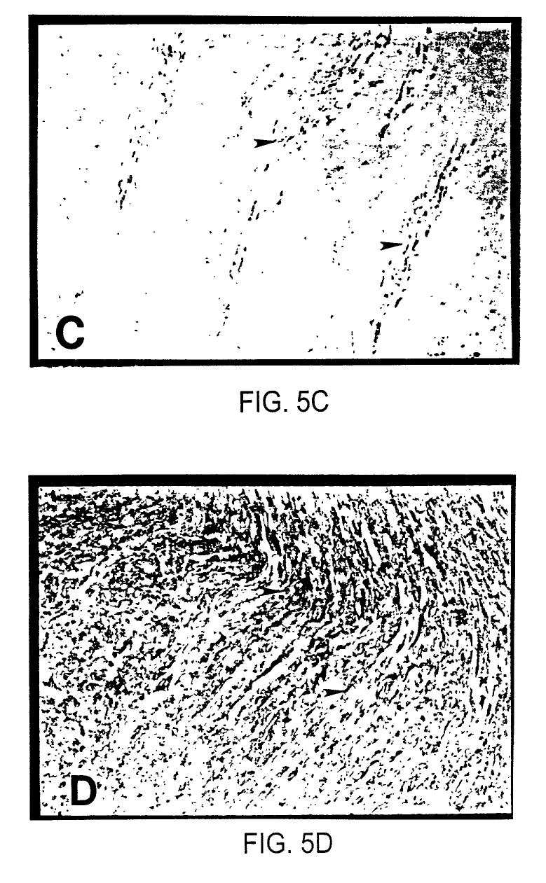

FIG. 5 demonstrates the presence of PTI-HS7 immunoreactivity localized to amyloid deposits from a case of AA amyloidosis. Shown in Figure A is Congo red staining for amyloid deposits in kidney glomeruli (arrows). Figure B demonstrates strong PTI-HS7 immunoreactivity in the kidney glomeruli containing AA amyloid deposits (arrows). Figure C shows Congo red birefringence staining in kidney medullary rays from another patient who was diagnosed with AA amyloidosis (arrowheads). An adjacent section immunostaining with PTI-HS7 demonstrates positive immunostaining to the same medullary ray amyloid deposits (arrowheads).

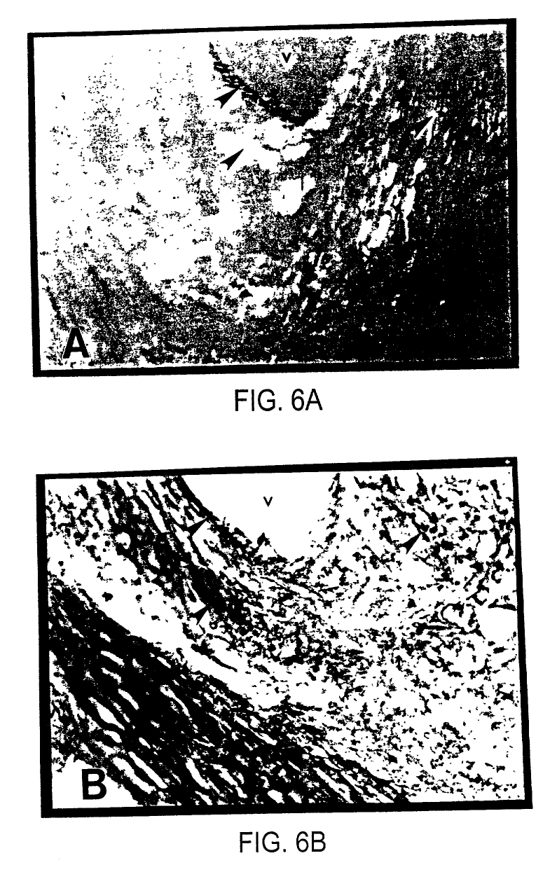

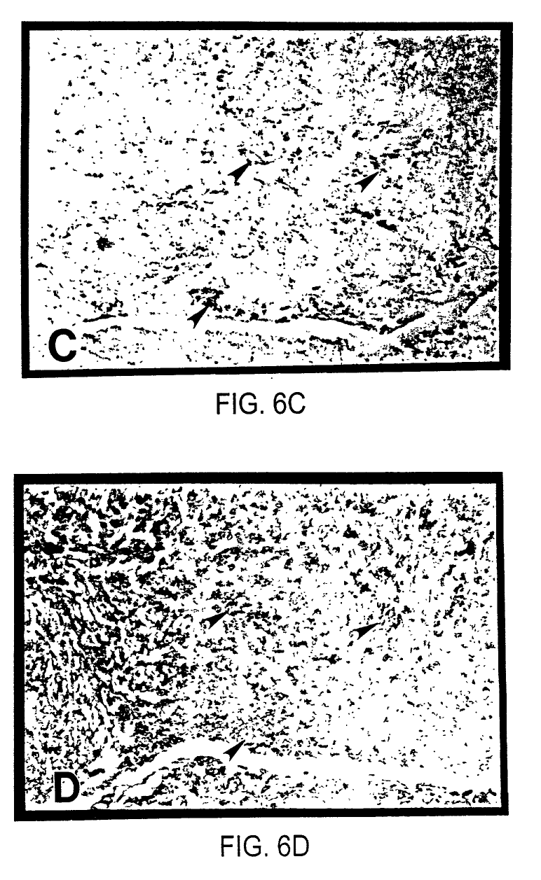

FIG. 6 demonstrates the presence of PTI-HS7 immunoreactivity localized to amyloid deposits from a case of AL and beta₂-microglobulin amyloidosis. As shown in Figure A, Congo red staining in heart depicts the presence of amyloid from a case of AL amyloidosis (arrowheads). Figure B depicts a serial section demonstrating the presence of PTI-HS7 immunoreactivity in the same locations as amyloid deposits (arrowheads). The vessel in Figures A and B is marked as “v”. Figure C demonstrates massive amyloid deposition in tendon (arrowheads) of a patient who had confirmed beta₂-microglobulin amyloidosis. Figure D demonstrates strong immunoreactivity with the same PTI-HS7 antibody in this tissue (arrowheads).

Antibodies The term “antibody” is meant to include polyclonal antibodies, monoclonal antibodies, chimeric antibodies, anti-idiotypic antibodies to antibodies specific for PTI-HS7 antigen of the present invention, as well as fragments thereof.

Polyclonal antibodies are heterogeneous populations of antibody molecules derived from the sera of animals immunized with an antigen.

A monoclonal antibody contains a substantially homogeneous population of antibodies specific to antigens, which population contains substantially similar epitope binding sites. For preparation of monoclonal antibodies, any technique which provides antibodies produced by continuous cell line cultures can be used. Examples include the hybridoma technique (Kohler and Milstein, Nature 256:495-497, 1975), the trioma technique, the human B-cell hybridoma technique (Kozbor et al, Immunology-Today 4:72, 1983), and the EBV-hybridoma technique to produce human monoclonal antibodies (Cole et al, in Monoclonal Antibodies and Cancer Therapy, Alan R. Liss, Inc., pp.77-96, 1985). Such antibodies may be of any immunoglobulin class including IgG, IgM, IgE, IgA, GILD and any subclass thereof.

Chimeric antibodies are molecules different portions of which are derived from different animal species, such as those having variable region derived from a murine monoclonal antibody and a human immunoglobulin constant region, which are primarily used to reduce immunogenicity in application and to increase yields in production. Chimeric antibodies and methods for their production are known in the art (ex. Cabilly et al, Proc. Natl. Acad. Sci. U.S.A 81:3273-3277, 1984; Harlow and Lane: Antibodies: A Laboratory Manual, Cold Spring Harbor Laboratory 1988).

An anti-idiotypic antibody is an antibody which recognizes unique determinants generally associated with the antigen-binding site of an antibody. An anti-idiotypic antibody can be prepared by immunizing an animal of the same species and genetic type (e.g., mouse strain) as the source of the monoclonal antibody with the monoclonal antibody to which an anti-idiotypic antibody is being prepared. The immunized animal will recognize and respond to the idiotypic determinants of the immunizing antibody by producing an antibody to these idiotypic determinants (the anti-idiotypic antibody). See, for example, U.S. Pat. No. 4,699,880, which is herein incorporated by reference.

The term “antibody” is also meant to include both intact molecules as well as fragments thereof, such as, for example, Fab and F(ab′)2, which are capable of binding antigen. Fab and F(ab′)2 fragments lack the Fc fragment of intact antibody, clear more rapidly from the circulation, and may have less non-specific tissue binding than an intact antibody (Wahl et al, J. Nucl. Med. 24:316-325, 1983).

The antibodies or fragments of antibodies, useful in the present invention may be used to quantitatively or qualitatively detect the PTI-HS7 antigen in a sample or to detect presence of cells which express the PTI-HS7 antigen of the present invention. This can be accomplished by immunofluorescence techniques employing a flourescently labeled antibody coupled with light microscopic, flow cytometric or fluorometric detection.

One of the ways in which a PTI-HS7 antibody can be detectably labeled is by linking the same to an enzyme and use in an enzyme immunoassay (EIA). This enzyme, in turn, when later exposed to an appropriate substrate, will react with the substrate in such a manner as to produce a chemical moiety which can be detected, for example, by spectrophotometric, fluorometric, or by visual means. Enzymes which can be used detectably label the antibody include, but are not limited to, malate dehydrogenase, staphylococcal nuclease, delta-5-steroid isomerase, yeast alcohol dehydrogenase, alpha glycerophosphate dehydrogenase, triose phosphate isomerase, horseradish peroxidase, alkaline phosphatase, asparaginase, glucose oxidase, betagalactosidase, ribonuclease, urease, catalase, glucose-6-phosphate dehydrogenase, glucoamylase and acetylcholinesterase. The detection can be accomplished by colormetric methods which employ a chromogenic substrate for the enzyme. Detection can be accomplished by calorimetric methods which employ a chromogenic substrate for the enzyme. Detection can also be accomplished by visual comparison of the extent of enzymatic reaction of a substrate with similarly prepared standards (see Harlow and Lane, Antibodies: A Laboratory Manual, Cold Spring Harbor Laboratory 1988; Ausubel et al, eds., Current Protocols in Molecular Biology, Wiley Interscience, N.Y. 1987, 1992).

Detection may be accomplished using any of a variety of other immunoassays. For example, by radiolabeling of the antibodies or antibody fragments, it is possible to detect R PTPase through the use of a radioimmunoassay (RIA). A good description of RIA may be found in Laboratory Techniques and Biochemistry in Molecular Biology, by Work et al, North Holland Publishing Company, NY (1978) with particular reference to the chapter entitled “An Introduction to Radioimmune Assay and Related Techniques” by Chard, incorporated entirely by reference herein. The radioactive isotope can be detected by such means as the use of a gamma-counter, a scintillation counter or by autoradiography.

It is also possible to label a PTI-HS7 antibody with a fluorescent compound, When the flourescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine, commercially available, e.g., from Molecular Probes, Inc. (Eugene, Oreg., U.S.A.).

The antibody can also be detectably labeled using fluorescence emitting metals such as 152EU, or other of the lanthanide series. These metals can be attached to the antibody using such metal groups as diethylenetriamine pentacetic acid (EDTA).

The antibody can also be detectably labeled by coupling it to a chemiluminescent compound. The presence of the chemiluminescent-tagged antibody is then determined by detecting the presence of luminescence that arises during the course of a chemical reaction, Examples of particularly useful chemiluminescent labeling compounds are luminol, isoluminol, theromatic acridinium ester, imidazole, acridinium salt, and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The antibodies (or fragments thereof) useful in the present invention may be employed histologically, as in immunofluorescence or immunoelectron microscopy, for in situ detection of a PTI-HS7 antigen of the present invention. In situ detection may be accomplished by removing a histological specimen from a patient, and providing the labeled antibody of the present invention to such a specimen. The antibody (or fragment) is preferably provided by applying or by overlaying the labeled antibody (or fragment) to a biological sample. Through the use of such a procedure, it is possible to determine not only the presence of PTI-HS7 antigen but also its distribution on the examined tissue. Using the present invention, those of ordinary skill will readily perceive that any of a wide variety of histological methods (such as staining procedures) can be modified in order to achieve such in situ detection.

In accordance with yet a further aspect of the present invention there is the PTI-HS7 antibody. These antibodies can be used for a number of important diagnostic and/or therapeutic applications as described herein. In one aspect of the invention, the PTI-HS7 antibody may be utilized for Western blot analysis (using standard Western blotting techniques knowledgeable to those skilled in the art) to detect the presence of the PTI-HS7 antigen in human tissues and in tissues of other species. In addition, Western blotting following by scanning densitometry (knowledgeable to those skilled in the art) can be used to quantitate and compare levels of the PTI-HS7 antigen in tissue samples or biopsies obtained from individuals with specific diseases (such as the amyloid diseases) in comparison to tissue samples or biopsies obtained from normal individuals or controls.

In yet another aspect of the invention, monoclonal antibodies made against the PTI-HS7 antigen can be utilized for immunoprecipitation studies (using standard immunoprecipitation techniques knowledgeable to one skilled in the art) to detect the PTI-HS7 antigen in tissues, cells and/or biological fluids. Use of the PTI-HS7 antibodies for immunoprecipitation studies can also be quantitative to determine relative levels of PTI-HS7 antigen in tissues, cells and/or biological fluids. Quantitative immunoprecipitation can be used to compare levels of the PTI-HS7 antigen in tissue samples or biopsies obtained from individuals with specific diseases (such as the amyloid diseases) in comparison to tissue samples or biopsies obtained from normal individuals or controls.

Diagnostic Applications

Use of PTI-HS7 Antibodies

Another aspect of the invention is to provide monoclonal PTI-HS7 antibodies which would be utilized to specifically detect heparan sulfate and associated amyloid deposits in human tissues and/or biological fluids. In a preferred embodiment, monoclonal PTI-HS7 antibodies, can be used to detect and quantify heparan sulfate and associated amyloid in human tissues and/or biological fluids. In a preferred embodiment, the anti-HS7 antibody can be utilized to detect heparan sulfate and associated amyloid deposits in biological fluids, and would have both diagnostic and therapeutic applications (described herein).

For detection of heparan sulfate and associated amyloid deposits described above in human tissues, cells, and/or in cell culture, the polyclonal and/or monoclonal antibodies can be utilized using standard immunohistochemical and immunocytochemical techniques, knowledgeable to one skilled in the art.

For detection and quantization of heparan sulfate PTI-HS7 antigen in biological fluids, including cerebrospinal fluid, blood, plasma, serum, urine, sputum, and/or stool, various types of ELISA assays can be utilized, knowledgeable to one skilled in the art. An antibody molecule of the present invention may be adapted for utilization in an immunometric assay, also known as a “two-site” or “sandwich” assay. In a typical immunometric assay, a quantity of unlabeled antibody (or fragment of antibody) is bound to a solid support or carrier, and a quantity of detectable labeled soluble antibody is added to permit detection and/or quantization of the ternary complex formed between solid-phase antibody, antigen, and labeled antibody.

In a preferred embodiment, a “sandwich” type of ELISA can be used. Using this preferred method a pilot study is first implemented to determine the quantity of binding of the PTI-HS7 antibody monoclonal antibody to microtiter wells. Once this is determined, aliquots (usually in 40 μl of TBS; pH 7.4) of the PTI-HS7 antibody are allowed to bind overnight to microtiter wells (Maxisorb C plate from Nunc) at 4° C. A series of blank wells not containing any PTI-HS7 antibody are also utilized as controls. The next day, non bound monoclonal antibody is shaken off the microtiter wells. All of the microtiter wells (including the blank wells) are then blocked by incubating for 2 hours with 300 μl of Tris buffered saline containing 0.05% Tween-20 (TTBS) plus 2% bovine serum albumin, followed by 5 rinses with TTBS. 200 μl of cerebrospinal fluid, blood, plasma, serum, urine, sputum, and/or stool and/or any other type of biological sample is then diluted in TTBS containing 2% bovine serum albumin and placed in wells (in triplicate) containing bound PTI-HS7 antibody (or blank) and incubated for 2 hours at room temperature. This dilution is done in a manner to be empirically derived, and which will be readily apparent to those skilled in the art. The dilution may be alternately done with either the antibody or the sample fluid.

The wells are then washed 5 times with TTBS. A second biotinylated-monoclonal antibody against the same PTI-HS7 antibody (but which is against a different epitope) is then added to each well (usually in 40 μl of TBS; pH 7.4) and allowed to bind for 2 hours at room temperature to any PTI-HS7 antigen captured by the first antibody. Following incubation, the wells are washed 5 times with TTBS.

Bound materials are then detected by incubating with 100 μl of peroxidase-avidin complex (1:250 dilution in TTBS with 0.1% BSA) for 1 hour on a rotary shaker. After 5 washes with TTBS, a substrate solution (100 μl, OPD-Sigma Fast from Sigma Chemical Co., St. Louis, Mo., USA) is added and allowed to develop significant color (usually 8-10 minutes). The reaction is stopped with 50 μl of 4N sulfuric acid and read on a standard spectrophotometer at 490 nm. This ELISA can be utilized to determine differences in PTI-HS7 antigen in biological fluids which can serve as a diagnostic marker to follow the progression on a live patient during the progression of disease (i.e. monitoring of amyloid disease as an example). In addition, quantitative changes in PTI-HS7 antigen can also serve as a prognostic indicator monitoring how a live patient will respond to treatment which targets a given amyloid disease. Such assays can be provided in a kit form.

Various ELISA assay systems, knowledgeable to those skilled in the art, can be used to accurately monitor the degree of PTI-HS7 antigen indicative of the level of associated amyloid deposits in nearby tissues, in biological fluids as a potential diagnostic indicator and prognostic marker for patients during the progression of disease (i.e. monitoring of an amyloid disease for example). Such assays can be provided in a kit form.

Other diagnostic methods utilizing the invention include diagnostic assays for measuring altered levels of PTI-HS7 antigen in various tissues compared to normal control tissue samples. Assays used to detect levels of PTI-HS7 antigen in a sample derived from a host are well-known to those skilled in the art and included radioimmunoassays, competitive-binding assays, Western blot analysis and preferably ELISA assays (as described above).

Yet another aspect of the present invention is to use the PTI-HS7 antibodies for labelings, for example, with a radionucleotide, for radioimaging or radioguided surgery, for in vivo diagnosis, and/or for in vitro diagnosis. In one preferred embodiment, radiolabeled nucleic acids or PTI-HS7 antibodies may be used as minimally invasive techniques to locate heparan sulfate and concurrent amyloid deposits in a living patient. These same imaging techniques could then be used at regular intervals (i.e. every 6 months) to monitor the progression of the amyloid disease by following the specific levels of the heparan sulfate detected by the PTI-HS7 antibody

Therapeutic Applications

Use of PTI-HS7 Antibodies

Yet another aspect of the present invention is to make use of PTI-HS7 antibodies as potential blocking therapeutics for the interaction of heparan sulfate and amyloid proteins in a number of biological processes and diseases (such as in the amyloid diseases described above). In a preferred embodiment, the PTI-HS7 antibody may be used to block the interaction of heparan sulfate-amyloid protein interactions, which is necessary for the formation, deposition, accumulation and persistence of amyloid deposits. Likewise, in another preferred embodiment PTI-HS7 antibodies may be given to a human patient as potential blocking antibodies to disrupt continued amyloid formation, deposition, accumulation and/or persistence in the given patient.

Preparations of PTI-HS7 antibodies for parenteral administration include sterile aqueous or non-aqueous solutions, suspensions, and emulsions, which may contain auxiliary agents or excipients which are known in the art. Pharmaceutical compositions such as tablets, pills, tablets, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectable solutions, sterile packaged powders, can be prepared according to routine methods and are known in the art.

In yet another aspect of the invention, the PTI-HS7 antibodies may be used as an effective therapy to block heparan sulfate proteoglycan and/or glycosaminoglycan and hence amyloid formation, deposition, accumulation and/or persistence as observed in the amyloid diseases. For example, the invention includes a pharmaceutical composition for use in the treatment of amyloidoses comprising a pharmaceutically effective amount of a PTI-HS7 antibody and a pharmaceutically acceptable carrier. The compositions may contain the PTI-HS7 antibody, either unmodified, conjugated to a potentially therapeutic compound, conjugated to a second protein or protein portion or in a recombinant form (i.e. chimeric or bispecific PTI-HS7 antibody). The compositions may additionally include other antibodies or conjugates. The antibody compositions of the invention can be administered using conventional modes of administration including, but not limited to, topical, intravenous, intra-arterial, intraperitoneal, oral, intralymphatic or intramuscular. Intravenous administration is preferred. The compositions of the invention can be a variety of dosage forms, with the preferred form depending upon the mode of administration and the therapeutic application. Optimal dosage and modes of administration for an individual patient can readily be determined by conventional protocols.

For example, administration of such a PTI-HS7 antibody composition may be by various parenteral routes such as subcutaneous, intravenous, intradermal, intramuscular, intraperitoneal, intranasal, transdermal or buccal routes. Alternatively, or concurrently, administration may be by the oral route. Parenteral administration can be by bolus injection or by gradual perfusion over time.

A preferred mode of using a PTI-HS7 antibody pharmaceutical composition of the present invention is by oral administration or intravenous application.

A typical regimen for preventing, suppressing or treating heparan sulfate related pathologies, such as comprises administration of an effective amount of a PTI-HS7 antibody, administered over a period of one or several days, up to and including between one week and about 24 months.

It is understood that the dosage of the PTI-HS7 antibody of the present invention administered in vivo or in vitro will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

The total dose required for each treatment may be administered by multiple doses or in a single dose. A PTI-HS7 antibody may be administered alone or in conjunction with other therapeutics directed to heparan sulfate-amyloid protein related pathologies, such as Alzheimer's disease or amyloid diseases, as described herein.

Effective amounts of a PTI-HS7 antibody composition, which may also include a PTI-HS7 antibody, are about 0.01μg protein or carbohydrate (by weight) to about 100 mg/kg body weight, and preferably from about 10 μg protein or carbohydrate to about 50 mg/kg body weight, such as 0.05, 0.07, 0.09, 0.1, 0.5, 0.7, 0.9., 1, 2, 5, 10, 20, 25, 30, 40, 45, 50, 55, 60, 65, 70, 75, 80, 85, 90, 95 or 100 mg/kg.

Preparations for parenteral administration include sterile aqueous or nonaqueous solutions, suspensions, and emulsions, which may contain auxiliary agents or excipients which are known in the art. In addition, pharmaceutical composition may contain suitable pharmaceutically acceptable carriers, such as excipients, carriers and/or auxiliaries which facilitate processing of the active compounds into preparations which can be used pharmaceutically.

Pharmaceutical compositions comprising PTI-HS7 antibody may also include suitable solutions for administration intravenously, subcutaneously, dermally, orally, mucosally, rectally or may by injection or orally, and contain from about 0.01 to 99 percent, preferably about 20 to 75 percent of active component (i.e. antibody) together with the excipient. Pharmaceutical compositions for oral administration include pills, tablets, caplets, soft and hard gelatin capsules, lozenges, sachets, cachets, vegicaps, liquid drops, elixers, suspensions, emulsions, solutions, and syrups.

### INDUSTRIAL APPLICABILITY

A method is disclosed for diagnosing an amyloid disease, or a susceptibility to an amyloid disease, in a patient, where the disease is related to the levels of a PTI-HS7 antigen in a sample from the patient. Antibody diagnosis or therapy of the amyloid diseases including Alzheimer's disease, is virtually unknown. Using the disclosed methods it will now be more possible to establish the presence, susceptibility to, or progression of, the amyloid disease in the patient.

In compliance with the statute, the invention has been described in language more or less specific as to structural features. It is to be understood, however, that the invention is not limited to the specific features shown, since the means and construction shown comprise preferred forms of putting the invention into effect. The invention is, therefore, claimed in any of its forms or modifications within the legitimate and valid scope of the appended claims, appropriately interpreted in accordance with the doctrine of equivalents.

We claim:

1. A method comprising the step of using a specific heparan sulfate glycosaminoglycan antibody identified herein as PTI-HS7 to diagnose the presence of amyloid deposits, or Parkinson's disease, in a human patient.

2. A method for diagnosing an amyloid disease, or a susceptibility to an amyloid disease, in a patient, the disease related to the levels of a PTI-HS7 antigen, the method comprising the step of testing with a PTI-HS7 antibody for elevated levels of PTI-HS7 antigen in the patient, whereby any elevated levels of PTI-HS7 antigen are indicative of the presence, susceptibility to, or progression of, the amyloid disease in the patient.

3. The method of claim 2 wherein the amyloid disease has an associated amyloid and the amyloid disease is selected from the group of amyloid disease associated with Alzheimer's disease, Down's syndrome, hereditary cerebral hemorrhage with amyloidosis of the Dutch type, the amyloid disease associated with type II diabetes, the amyloid disease associated with chronic inflammation, various forms of malignancy and Familial Mediterranean Fever, the amyloid disease associated with multiple myeloma and other B-cell dyscrasias, the amyloid disease associated with the prion diseases including Creutzfeldt-Jakob disease, Gerstmann-Straussler syndrome, kuru and animal scrapie, the amyloid disease associated with long-term hemodialysis and carpal tunnel syndrome, the amyloid disease associated with endocrine tumors such as medullary carcinoma of the thyroid, and the alpha-synuclein associated diseases including Parkinson's disease and Lewy body disease.

4. The method of claim 3 wherein the amyloid disease is associated with Alzheimer's disease.

5. The method of claim 3 wherein the associated amyloid is beta-amyloid protein or Aβ, AA amyloid or inflammation-associated amyloid, AL amyloid, amylin or islet amyloid polypeptide, PrP amyloid, beta₂-microglobulin amyloid, transthyretin or prealbumin, or variants of procalcitonin.

6. The method of claim 2 wherein the testing with the PTI-HS7 antibody uses a biological fluid of the patient.

7. The method of claim 6 wherein the biological fluid is selected from the group of fluids consisting of blood, plasma, serum, cerebrospinal fluid, sputum, saliva, urine and stool.

8. The method of claim 2 wherein the testing with the PTI-HS7 antibody uses a tissue section of the patient by standard immunohistochemistry.

9. A method for the treatment of a patient having an identified clinical need to interfere with the pathological effects of amyloid deposits, the method comprising the step of administering to the patient a therapeutically effective amount of PTI-HS7 antibody.

10. A method for detection of amyloid disease in a human patient, the method comprising the step of using a radiolabeled PTI-HS7 antibody for radioimaging or in vivo diagnosis.

11. A method for detection and quantization of amyloid associated HS7 antigen in biological fluids comprising the steps of:
a) binding an anti-PTI-HS7 monoclonal antibody to a microtiter well;
b) blocking the microtiter well with Tris-buffered saline or the like containing detergent plus bovine serum albumin;
c) adding to the well a quantity of biological fluid selected from the group of fluids consisting of cerebrospinal fluid, blood, plasma, serum, urine, sputum, saliva, urine and stool;
d) incubating the biological fluid in the microtiter well to bind the antibody;
e) adding to the microtiter well a second labeled monoclonal antibody against the same HS7 antigen but which is against a different epitope to bind to any HS7 protein captured by the first antibody;
f) incubating with a substrate until a significant color reaction develops to detect antibody bound materials.

12. A method of treatment, prevention or management of an amyloidosis, or a disease related to alpha-synuclein, in a mammalian subject susceptible to, or afflicted by, the amyloidosis or alpha-synuclein disease, the method comprising the step of administering to the subject a therapeutic amount of PTI-HS7 antibody.

13. A method for diagnosing an amyloid disease in a subject, the method comprising the step of determining a level of PTI-HS7 antigen in a sample from the subject and comparing the level of PTI-HS7 antigen in the sample to normal levels, wherein an increase in the level of PTI-HS7 antigen in the sample relative to normal indicates the amyloid disease.

14. A method for determining whether a subject is at increased risk of developing an amyloid disease, or diagnosing or prognosing the amyloid disease in the subject, or monitoring a progression of the amyloid disease in the subject, the method comprising the steps of: a) determining a level of PTI-HS7 antigen in a sample from the subject; and b) comparing the level of PTI-HS7 antigen in the sample to a reference value representing a known disease or health status; thereby determining whether the subject is at increased risk of developing the amyloid disease, or diagnosing or prognosing the amyloid disease in the subject, or monitoring the progression of the amyloid disease in the subject.

15. A method of evaluating a treatment for an amyloid disease, the method comprising the step of determining a level of PTI-HS7 antigen in a sample from a subject under the treatment; and b) comparing the level of PTI-HS7 antigen in the sample to a reference value representing a known disease or health status; thereby evaluating the treatment for the amyloid disease.

16. The method of claim 14, wherein the sample is a biological fluid, preferably cerebrospinal fluid.

17. The method of claim 14, wherein an increase of level of PTI-HS7 antigen in the sample from the subject relative to a reference value representing a known amyloid disease or health status indicates the diagnosis or prognosis, or progression, or increased risk of the amyloid disease.

18. The method of claim 14 wherein the level of PTI-HS7 antigen in the sample is detected and quantified using an immunoassay and/or a binding assay.

19. The method of claim 14, wherein the reference value is that of a level of PTI-HS7 antigen in a sample from a subject not afflicted with the amyloid disease.

20. The method of claim 14, further comprising comparing the level of PTI-HS7 antigen in the sample from the subject with a level of PTI-HS7 antigen in a series of samples from the subject taken over a period of time.

21. A kit for determining increased risk of developing an amyloid disease, or diagnosing or prognosing the amyloid disease in a subject, or monitoring a progression of the amyloid disease in the subject, the kit comprising:
a) a reagent for detecting the presence or absence of PTI-HS7 antigen, the reagent comprising PTI-HS7 antibody, and
b) instructions for determining increased risk of developing an amyloid disease, or diagnosing or prognosing the amyloid disease in a subject, or monitoring a progression of the amyloid disease in the subject by detecting and/or quantifying a level of PTI-HS7 antigen in a sample from a subject, and determining whether the subject is at increased risk of developing the amyloid disease, or diagnosing or prognosing the amyloid disease in a subject, or monitoring the progression of the amyloid disease in the subject, wherein a varied level of PTI-HS7 antigen in a sample relative to a reference value representing a known disease or health status is indicates the increased risk of developing an amyloid disease, or the diagnosis or prognosis of the amyloid disease in the subject, or the progression of the amyloid disease in the subject.
